(19) United States
(12) Patent Application Publication
(10) Pub. No.: US 2025/0281766 A1
(43) Pub. Date: Sep. 11, 2025

(54) CARDIAC STIMULATION DEVICE

(71) Applicant: NATIONAL TSING HUA UNIVERSITY, Hsinchu City (TW)

(72) Inventors: TING-WEI WANG, Hsinchu City (TW); EN-ZHU LYU, Hsinchu City (TW)

(73) Assignee: NATIONAL TSING HUA UNIVERSITY, Hsinchu City (TW)

(21) Appl. No.: 18/971,980

(22) Filed: Dec. 6, 2024

(30) Foreign Application Priority Data
Mar. 6, 2024 (TW) 113108122

(51) Int. Cl. A61N 1/40; A61N 1/365

(52) U.S. Cl. CPC A61N 1/40; A61N 1/365

(57) ABSTRACT

The cardiac stimulation device includes a sensing module and a stimulation module. The sensing module is configured to measure at least one cardiac state of a subject. The stimulation module is coupled to the sensing module. The stimulation module includes a stimulation emitting coil, a stimulation control circuit and a stimulation receiver. The stimulation control circuit is coupled to the stimulation emitting coil. The stimulation control circuit is configured to drive the stimulation emitting coil to emit a stimulation electromagnetic signal based on the at least one cardiac state. The stimulation receiver includes a stimulation receiving coil and a stimulation electrode. The stimulation receiving coil is configured to receive the stimulation electromagnetic signal to generate a stimulation voltage provided to the stimulation electrode. The stimulation electrode is arranged at a cardiac stimulation area of the subject. The stimulation voltage is provided to the cardiac stimulation area through the stimulation electrode.

### FIELD OF THE INVENTION

The present invention relates to a cardiac stimulation device; in particular, a cardiac stimulation device which is configured to stimulate the heart with voltage through an electromagnetic induction.

### BACKGROUND OF THE INVENTION

For patients suffer from abnormal heart rhythm, cardiac stimulation devices for regulating heart rhythm can effectively manage and regulate the patient's heart rhythm. In general, conventional cardiac stimulation devices provide auxiliary stimulation to patients' heart by monitoring their cardiac state in real-time. For example, if detected an abnormal cardiac state such as arrhythmia, bradycardia, or atrial fibrillation, the cardiac stimulation device will immediately provide electrical stimulation to the patient's heart. The cardiac stimulation device provides a preventive effect on atrial fibrillation caused by partial bradycardia, and reduces the frequency or duration for happening symptoms. Therefore, the cardiac stimulation device improves the quality of life of patients and avoiding the risk of related heart diseases or fatal heart disease attacks.

However, the conventional cardiac stimulation devices require a persistent and uninterrupted power supply to ensure a long-term continuous operation. When the cardiac stimulation device fails to operate stably for a long time, it will greatly affect the patient's life. Although a long-lasting battery set and/or a low-power configuration will extend the durability of the cardiac stimulation devices, the cardiac stimulation device can only operate stably for about ten years at most, and the battery installed inside the cardiac stimulation device still need to be replaced to extend the service time before the expiration date. In terms of battery replacement, patients must take the risk for the secondary surgery to replace the battery, which not only increases the risk of infection, but also has incalculable effects on the patient's physical and mental health during or after the secondary surgery. In addition, multiple surgeries will also increase the medical expenses that patients need to bear.

In addition, the conventional cardiac stimulation devices still face many hardware challenges that need to be overcome or optimized. For example, the built-in battery may result in a larger volume, and the structural rigidity or additional weight of the conventional cardiac stimulation device may cause discomfort to the subject.

Accordingly, there are still issues for improving in the field, such as battery life and hardware, that need to be overcome and resolved in the cardiac stimulation devices applied for regulating heart rhythm.

### SUMMARY OF THE INVENTION

Therefore, the present invention provides a cardiac stimulation device to effectively solve the problems encountered in the conventional cardiac stimulation devices.

One of the objects of the present invention is to provide a cardiac stimulation device that does not require battery replacement, which reducing the inconvenience of the secondary surgery for battery replacement.

One of the objects of the present invention is to provide a cardiac stimulation device providing a better wearing experience and less hardware limitations to patients than the conventional cardiac stimulation device.

The present invention provides a cardiac stimulation device. The cardiac stimulation device includes a sensing module and a stimulation module. The sensing module is configured to measure at least one cardiac state of a subject. The stimulation module is coupled to the sensing module. The stimulation module includes a stimulation emitting coil, a stimulation control circuit and a stimulation receiver. The stimulation control circuit is coupled to the stimulation emitting coil. Wherein the stimulation control circuit is configured to drive the stimulation emitting coil to emit a stimulation electromagnetic signal based on the at least one cardiac state. The stimulation receiver includes a stimulation receiving coil and a stimulation electrode. Wherein the stimulation receiving coil is configured to receive the stimulation electromagnetic signal to generate a stimulation voltage provided to the stimulation electrode. Wherein the stimulation electrode is arranged at a cardiac stimulation area of the subject. And wherein the stimulation voltage is provided to the cardiac stimulation area through the stimulation electrode.

In an embodiment, the sensing module further includes a sensing emitting coil and a sensing electrode, wherein the sensing receiving coil is coupled to the sensing electrode, and configured to receive the sensing electromagnetic signal and generate the feedback electromagnetic signal. Wherein the sensing electrode is arranged at a cardiac sensing area of the subject. And wherein the sensing electrode is configured to adjust at least one electrical parameter of the sensing receiving coil for generating the feedback electromagnetic signal based on the at least one cardiac state.

In an embodiment, the sensing electrode includes a first electrode and a second electrode; and wherein a gap is formed between the first electrode and the second electrode, and the gap is varied by the at least one cardiac state.

In an embodiment, the first electrode and the second electrode form an interdigital electrode.

In an embodiment, the sensing control circuit includes a frequency adjustment unit coupled to the sensing emitting coil; and wherein frequency adjustment unit is configured to adjust a frequency of the sensing electromagnetic signal.

In an embodiment, the frequency adjustment unit includes a capacitor array configured to adjust an impedance of the sensing emitting coil.

In an embodiment, the sensing module further includes a shielding component at least arranged at a first side of the sensing emitting coil. Wherein the shielding component at least shields a portion of the sensing electromagnetic signal emitted toward to a first direction.

In an embodiment, the stimulation control circuit includes a discharge controller, a discharge controller configured to provide a control signal based on the at least one cardiac state, a storage capacitor configured to storage a driving voltage, and a switch configured to receive the control signal and enable transmitting the driving voltage to the stimulation emitting coil based on the control signal.

In an embodiment, the stimulation control circuit further includes a boost circuit configured to receive a supply voltage and boost the supplied power to the driving voltage.

In an embodiment, the cardiac stimulation device further includes a control module configured to receive the at least one cardiac state and provide a control signal to the stimulation module. Wherein the stimulation module is configured to emit the stimulation electromagnetic signal based on the control signal

In summary, the cardiac stimulation device of the present invention has a stimulation module. The stimulation module provides stimulation electromagnetic signal from outside to the subject's body to generate a stimulation voltage through the stimulation receiving coil by receiving the stimulation electromagnetic signal. The stimulation voltage is provided by a stimulation electrode and used to stimulate a specific cardiac area of the subject by connecting the stimulation electrode to the stimulation receiving coil and fixing the stimulation electrode at the specific cardiac area. The stimulation module of the cardiac stimulation device provides the function of cardiac regulation. In addition, the cardiac stimulation device of the present invention measures the cardiac state through the sensing module and wirelessly and remotely deliver the stimulation voltage required for cardiac stimulation and regulation through the stimulation module. Accordingly, the cardiac stimulation device achieves an implantable cardiac stimulation in the patient's body without installing any batteries.

### BRIEF DESCRIPTION OF THE DRAWINGS

The accompanying drawings are presented to aid in the description of various aspects of the disclosure and are provided solely for illustration of the aspects. To simplify the drawings and highlight the contents to be presented in the drawings, the well-known structures or elements in the drawings may be drawn in a simple schematic manner or presented in an omitted manner. For example, the number of elements may be singular or plural. These drawings are provided only to explain these aspects and not to limit thereof.

FIG. 1 is a schematic diagram of the cardiac stimulation device according to an embodiment of the present invention.

FIGS. 2A to 2B are block diagrams of the stimulation module according to an embodiment of the present invention.

FIG. 3 is a schematic diagram of a cardiac stimulation device with a control module according to an embodiment of the present invention.

FIG. 4 is a schematic diagram of a sensing module using eddy current induction according to one embodiment of the present invention.

FIG. 5 is a schematic diagram of a sensing receiving coil and a sensing electrode according to an embodiment of the present invention.

FIGS. 6A and 6B are schematic diagrams of different settings of the sensing electrodes according to an embodiment of the present invention.

### DETAILED DESCRIPTION OF THE PREFERRED EMBODIMENTS

Even though the terms such as “first”, “second”, and “third” may be used to describe an element, a part, a region, a layer, and/or a portion in the present specification, these elements, parts, regions, layers and/or portions are not limited by such terms. Such terms are used to differentiate an element, a part, a region, a layer, and/or a portion from another element, part, region, layer, and/or portion. Therefore, in the following discussions, a first element, portion, region, or portion may be called a second element, portion, region, layer, or portion, and do not depart from the teaching of the present disclosure. The terms “comprise,” “include”, or “have” used in the present specification are open-ended terms and mean to “include”, but not limited to

As used herein, the term “coupled to” in the various tenses of the verb “couple” may mean that element A is directly connected to element B or that other elements may be connected between elements A and B (i.e., that element A is indirectly connected with element B).

The terms “approximate” or “essentially” used in the present specification include the value itself and the average values within the acceptable range of deviation of the specific values confirmed by a person having ordinary skill in the current art, considering the specific measurement discussed and the number of errors related to such measurement (that is, the limitation of the measurement system). For example, “about” may mean within one or more standard deviations of the value itself or +30%, +20%, +10%, +5%. In addition, “about”, “approximate”, or “essentially” used in the present specification may select a more acceptable range of deviation or standard deviation based on optical property, etching property, or other properties. One cannot apply one standard deviation to all properties.

The present invention provides a cardiac stimulation device. The cardiac stimulation device includes a sensing module configured to measure at least one cardiac state of a subject, and a stimulation module coupled to the sensing module. The stimulation module includes a stimulation emitting coil, a stimulation control circuit coupled to the stimulation emitting coil, and a stimulation receiver having a stimulation receiving coil and a stimulation electrode. The stimulation module provides the stimulation electromagnetic signal from the outside of the subject's body. The stimulation receiving coil receives the stimulation electromagnetic signal, and coverts the stimulation electromagnetic signal to a stimulation power by magneto-electric effect. The stimulation power is delivered by the stimulation electrode arranged at the subject's heart to stimulate a specific cardiac area of the subject's heart and regulate the cardiac state. The stimulation power used for stimulating is wirelessly delivered to the specific cardiac area through the stimulation module for cardiac stimulation. Therefore, the cardiac stimulation device achieves implantable cardiac stimulation in subject's body without the need to install any batteries inside the subject's body.

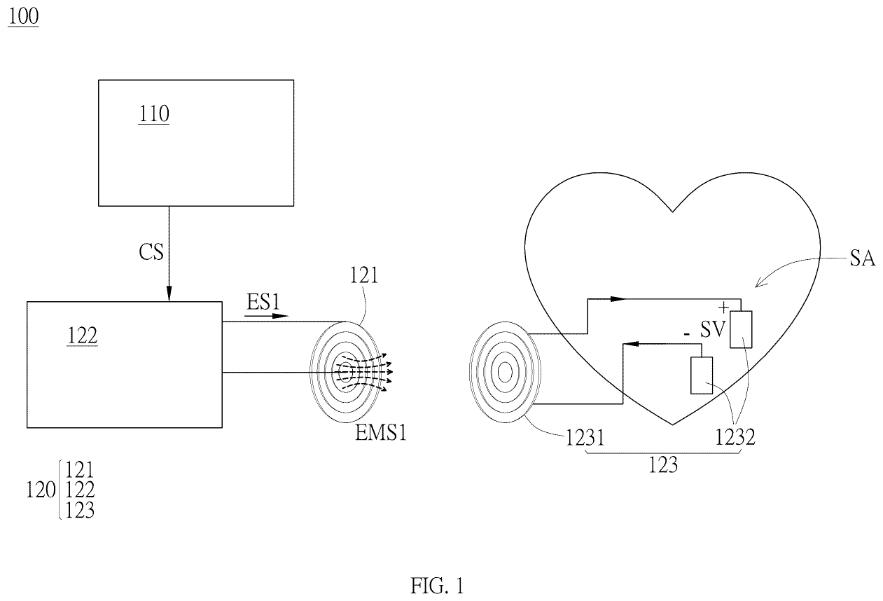

Specifically, referring to FIG. 1, which illustrates that a cardiac stimulation device 100 includes a sensing module 110 configured to measure at least one cardiac state (CS) of a subject, and a stimulation module 120 coupled to the sensing module 110. The stimulation module 120 includes a stimulation emitting coil 121, a stimulation control circuit 122 coupled to the stimulation emitting coil 121, and a stimulation receiver 123 having a stimulation receiving coil 1231 and a stimulation electrode 1232. The stimulation control circuit 122 is configured to drive the stimulation emitting coil 121 to emit a stimulation electromagnetic signal (MS1) based on the at least one cardiac state (CS). The stimulation receiving coil 1231 receives the stimulation electromagnetic signal (MS1) and generates a stimulation voltage (SV) provided to the stimulation electrode 1232. The stimulation electrode 1232 is arranged at a cardiac stimulation area (SA) of the subject. The stimulation voltage (SV) is provided to the cardiac stimulation area (SA) through the stimulation electrode 1232.

The stimulation module 120 is configured to stimulate the heart of the subject by using electromagnetic induction. The stimulation module 120 may be divided into two main parts. One of the two main parts includes the stimulation emitting coil 121 arranged outside the subject's body (such as skin surface) and the stimulation control circuit 122 coupled to the stimulation emitting coil 121, and the other includes the stimulation receiver 123 arranged on the subject or implanted in the subject. The stimulation control circuit 122 provides a stimulation electrical signal (ES1) (preferably a pulse electrical signal) to the stimulation emitting coil 121. The stimulation emitting coil 121 converts the stimulation electrical signal (ES1) into a stimulation electromagnetic signal (EMS1) based on the electromagnetic effect and provides the stimulation electromagnetic signal (EMS1) to the stimulation receiving coil 1231 of the stimulation receiver 123. The stimulation receiving coil 1231 converts the stimulation electromagnetic signal (EMS1) into the stimulation voltage (SV) based on to the magneto-electric effect and provides the stimulation voltage (SV) to the stimulation electrode 1232.

The stimulation control circuit 122 is coupled to the sensing module 110 and receives signals represented as the at least one cardiac state (CS) measured by the sensing module 110. The cardiac state (CS) referred to in the present invention is, for example, the cardiac operating state observed by measuring cardiac electrical signals, atrial or ventricular blood flow signals, pulse signals, or other cardiac state (CS) indicators through the sensing module 110. When the at least one cardiac state (CS) of the subject reaches the level where the heart rhythm needs to be adjusted or regulated, the stimulation control circuit 122 will output the stimulation electrical signal (ES1) for subsequent cardiac stimulation. After stimulation, the subject's cardiac state (CS) is continuously observed by the sensing module 110, and the stimulation control circuit 122 will evaluate whether to continue the next stimulation or complete the stimulation according to the observed cardiac state (CS).

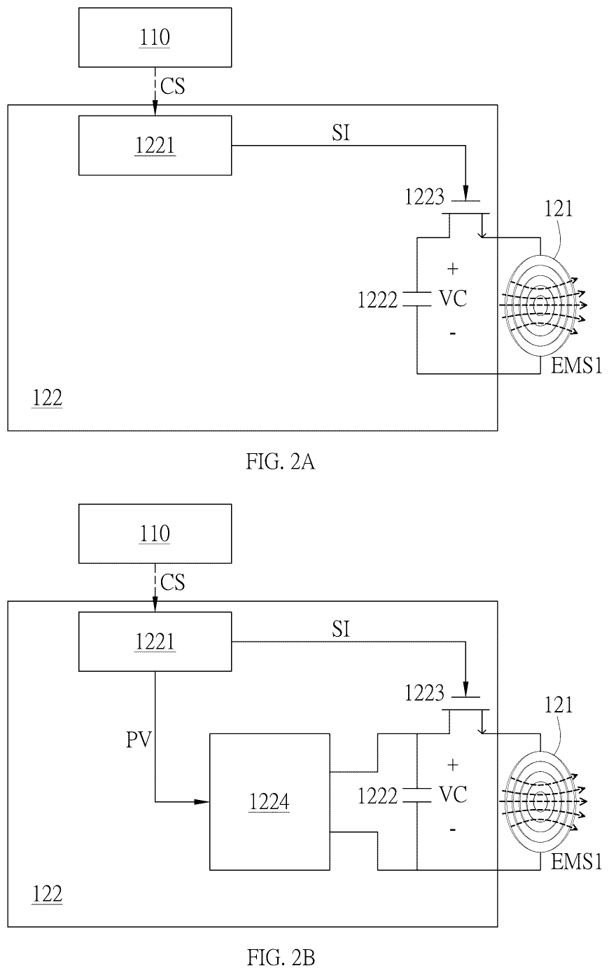

The stimulation control circuit 122 can be composed by any means that provides the stimulation electrical signal (ES1). In an embodiment, the stimulation control circuit 122 is preferably configured to provide pulse electrical signals. In the embodiment, referring to FIG. 2A, the stimulation control circuit 122 includes a discharge controller 1221, a storage capacitor 1222 and a switch 1223. The discharge controller 1221 is configured to provide a control signal based on the at least one cardiac state (CS). The storage capacitor 1222 is configured to store the driving voltage (Vc). The switch 1223 is configured to receive the control signal and transmit the driving voltage (Vc) to the stimulation emitting coil 121 based on the control signal. When the discharge controller 1221 (e.g. microcontroller) determined that the subject's heart requiring to be stimulated based on the current cardiac state (CS), the discharge controller 1221 will provide a stimulation instruction (SI) to the switch 1223 (e.g. transistor switch, or switch). After the switch 1223 is turned on, a conductive path is formed among the storage capacitor 1222 and the stimulation emitting coil 121. The driving voltage (Vc) stored in the storage capacitor 1222 can be provided to the stimulation emitting coil 121. The driving voltage (Vc) generates an RLC transient response on the stimulation emitting coil 121 to generate the stimulation electromagnetic signal (MS1). The current relationship between the storage capacitor 1222 and the stimulation emitting coil 121 during discharge can be expressed by the following formula:

$$I ( t ) = \frac{Vc}{ω L} {e}^{- α t} sin ω t$$

From the above formula, it can be known that the value of current I(t) is proportional to the driving voltage (Vc) and inversely proportional to the inductance value (L) of the stimulation emitting coil 121. Therefore, the current I(t) can be adjusted by adjusting the driving voltage (Vc), the inductance value (L) of the stimulation emitting coil 121, or the opening time of the switch 1223 to adjust the stimulation electromagnetic signal (EMS1) output by the stimulation emitting coil 121.

In an embodiment, referring to FIG. 2B, the stimulation control circuit 122 further includes a boost circuit 1224. The boost circuit 1224 is configured to receive a supply voltage and boost the supply voltage to the driving voltage (Vc). For example, the boost circuit 1224 can boost the supply voltage (PV) (e.g. 5V) supplied by an external power supply circuit. The boosted voltage (i.e. the driving voltage (Vc)) is stored by the storage capacitor 1222. In this way, the driving voltage (Vc) stored in the storage capacitor 1222 can be regulated or amplified by the boost circuit 1224, and lower supply voltages (such as the voltage provided by the discharge controller 1221) can also be used without the need for additional power supply. It should be noted that FIG. 2B illustrates only an example implementation means for the power supply of the boost circuit 1224 provided by the discharge controller 1221, the present invention is not limited to the example shown in FIG. 2B. In other words, the boost circuit 1224 can also derive power from other voltage sources.

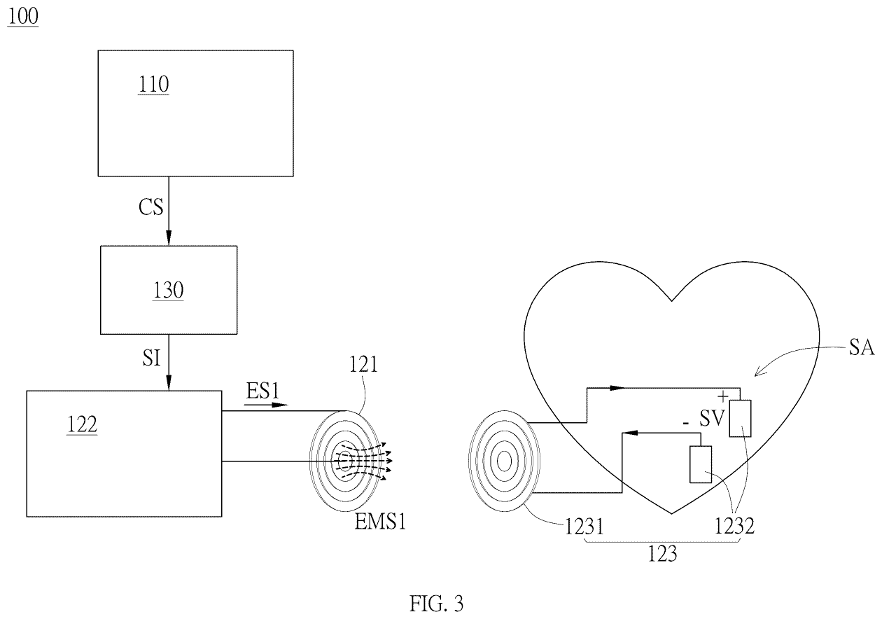

In an embodiment, referring to FIG. 3, the cardiac stimulation device 100 further includes a control module 130. The control module 130 is configured to receive the at least one cardiac state (CS) and provide a control signal to the stimulation module 120. The stimulation module 120 emits the stimulation electromagnetic signal (EMS1) based on the control signal. Specifically, the control module 130 is an independent component coupled to the stimulation module 120 and the sensing module 110. Hence, the control module 130 may be computing capable components such as microprocessors, FPGAs, smartphones, tablets, etc. It should be noted that the control module 130 shown in FIG. 3 is not mutually exclusive with the embodiments shown in FIG. 2A or FIG. 2B. For example, the control module 130 may be configured to provide the stimulation instruction (SI) to the discharge controller 1221 and control the switch 1223 through the discharge controller 1221. On the other hand, the control module 130 can also be configured to directly provide the stimulation instruction (SI) required by the switch 1223 to the switch 1223. Hence, the control module 130 directly controls the conductive path among the switch 1223 and the stimulation emitting coil 121. In addition, the control module 130 may be configured to provide the stimulation instruction (SI) to the stimulation module 120 based on the cardiac state (CS) provided by the sensing module 110. The control module 130 can be served as an intermediate medium between the sensing module 110 and the stimulation module 120. The control module 130 can be selected based on the computational complexity of the cardiac state (CS) to avoid mismatches caused by different signal types or computational requirements between the sensing module 110 and the stimulation module 120.

In an embodiment, the discharge controller 1221 or the control module 130 can be configured to adjust the activation frequency or duty cycle of the switch 1223 or the duration of a stimulation based on the current cardiac state (CS) of the subject or the feedback cardiac state (CS) after the stimulation. On the other hand, in an embodiment, the discharge controller 1221 or the control module 130 can also be configured to adjust the driving voltage (Vc) stored in the storage capacitor 1222. With the discharge controller 1221 or the control module 130, the cardiac stimulation device 100 can provide stimulation electromagnetic signals with different intensities, frequencies, or emitting modes, and achieve a proper or best stimulation effect for different subjects or different cardiovascular states.

The present invention is not limited to the formation of the stimulation emitting coil 121 and/or the stimulation receiving coil 1231. The stimulation emitting coil 121 and/or the stimulation receiving coil 1231 can be formed on a substrate (preferably a flexible substrate) through a conductor line(s) formed as a radiation path to emit the electromagnetic signals. In a further embodiment, the stimulation emitting coil 121 and/or the stimulation receiving coil 1231 can be formed as a single turn coil, a multi-turn coil, a spiral coil, a concentric circle coil, or any other means for radiating by enameled wires or conductors with or without the substrate. Moreover, the stimulation emitting coil 121 and the stimulation receiving coil 1231 are not limited to having the same coil parameters. For example, different turns can be selected to adjust the ratio of the stimulation electrical signal (ES1) to the stimulation voltage (SV), but not limited to this.

The present invention does not limit the arranging location of the stimulation receiving coil 1231. The stimulation receiving coil 1231 can be arranged on the skin, subcutaneously, on outside of the heart, or any locations suitable for receiving the stimulation electromagnetic signal (MS1). For example, the arranging location of the stimulation receiving coil 1231 may be refer to the arranging location for implantable defibrillators (ICD) in conventional technology to arrange at the subcutaneous position of the left anterior chest. The stimulation receiving coil 1231 is coupled to the stimulation electrode 1232 through a conductor line(s). Therefore, the stimulation electrode 1232 can be arranged on the cardiac stimulation area (SA) away or closed to the stimulation receiving coil 1231. The cardiac stimulation area (SA) can be any position of the subject's heart that can be stimulated, such as the arranging location for a conventional electrode setup of implantable defibrillators. With the stimulation electrode 1232, the cardiac stimulation device 100 can provide an electrical stimulation for the subject's heart and effectively control the rhythm or the ventricular rate of the subject's heart.

The pairing between the stimulation electrode 1232 and the stimulation receiving coil 1231 of the present invention is not limited to one-to-one. For example, a stimulation receiving coil 1231 may correspond to multiple stimulation electrodes 1232. Thus, the multiple stimulation electrodes 1232 can stimulate multiple position of the subject's heart simultaneously or in sequence. On the other hand, a plurality of the stimulation receiving coils 1231 can correspond to multiple stimulation electrodes 1232, so that each stimulation position can be independently provided with a specific stimulation voltage (SV). In addition, the stimulation emitting coil 121 corresponding to the stimulation receiving coil 1231 can also be configured in a one-to-one, many to one, or many to many. Through the above configuration, the selectivity of setting the stimulation position and stimulation electromagnetic signal (EMS1) can be improved. The stimulation emitting coil 121 can also limit the divergence of the stimulation electromagnetic signal (MS1) through a shielding component. For example, a shielding component can be set around the stimulation emitting coil 121 to block the stimulation electromagnetic signal (MS1) and reduce the risk of affecting other circuit components, medical devices, or other stimulation emitting coils 121 (if there are multiple stimulation emitting coils).

In summary, the stimulation receiving coil 1231 is configured to receive the stimulation electromagnetic signal (EMS1) from the stimulation emitting coil 121 to generate the stimulation voltage (SV). The stimulation electrode 1232 is configured to provide the stimulation voltage (SV) to the cardiac stimulation area (SA) of the subject's heart. The stimulation receiving coil 1231 and the stimulation electrode 1232 arranged on/in the subject are both composed by passive components operating without power supply. The generation of stimulation voltage (SV) is achieved through an external stimulation electromagnetic signal (EMS1), and the requirement of the battery or the power supply is limited to the stimulation module 120 and/or the sensing module 110 arranged outside the subject. Therefore, the subject is free to the need to replace the power supply or the battery for the stimulation receiving coil 1231 and the stimulation electrode 1232. It can effectively reduce the discomfort and risk of subject for replacing batteries inside the subject's body.

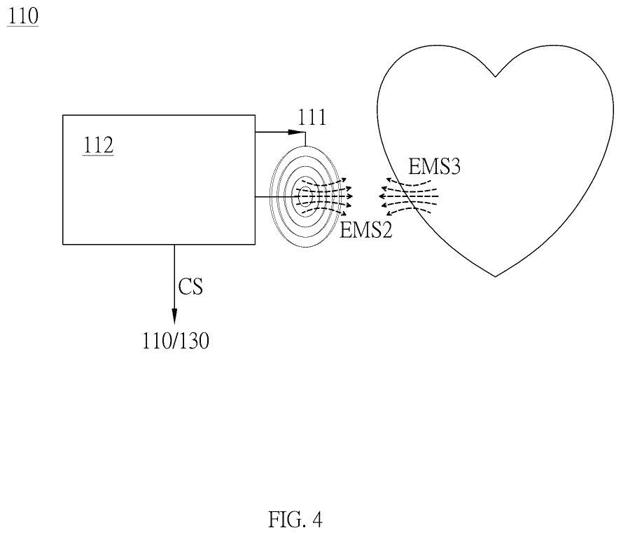

It should be noted that the sensing module 110 is not limited to the sensing mechanism. For example, the sensing module 110 may be configured to measure the at least one cardiac state (CS) of the subject by using principles such as acoustics, optics, electricity, or magnetism. In a specific embodiment, the sensing module 110 is configured to measure the at least one cardiac state (CS) through eddy current induction. Specifically, referring to FIG. 4, the sensing module 110 includes a sensing emitting coil 111 and a sensing control circuit 112 coupled to the sensing emitting coil 111. The sensing control circuit 112 is configured to drive the sensing emitting coil 111 to emit a sensing electromagnetic signal (EMS2) to the heart position of the subject, and receive a sensing signal, generated by a feedback electromagnetic signal (EMS3) from the heart position, from the sensing emitting coil 111. Wherein the feedback electromagnetic signal (EMS3) is induced by the sensing electromagnetic signal (EMS2) at the heart position. The sensing control circuit 112 is further configured to calculate and determine the at least one cardiac state (CS) based on the sensing signal.

In the embodiment, the sensing emitting coil 111 can be any radiator that can emit electromagnetic waves. The present invention does not limit the structure, shape, or material for the sensing emitting coil 111. The sensing control circuit 112 is configured to provide an AC signal to the sensing emitting coil 111 to cause the sensing emitting coil 111 to emit the sensing electromagnetic signal (EMS2). When the sensing electromagnetic signal (EMS2) is transmitted to the heart of the subject, the ionic liquids (such as blood inside the heart) can be considered as a planar conductor. When the planar conductor received the sensing electromagnetic signal (EMS2), the planar conductor will be induced and generate eddy currents. The generation of eddy current will generate the feedback electromagnetic signal (EMS3) at the planar conductor and feedback to the sensing emitting coil 111. Thus, the sensing emitting coil 111 receives the feedback electromagnetic signal (EMS3) and generates the sensing signal. It should be noted that the present invention is not limited to the form or type of the sensing signal. For example, the feedback electromagnetic signal (EMS3) causes a change in the measured inductance value of the sensing emitting coil 111, the measured inductance value can be considered as a sensing signal by measuring the change in inductance value of the sensing emitting coil 111. On the other hand, the frequency or amplitude of the feedback electromagnetic signal (EMS3), or the frequency or phase change between the sensing electromagnetic signal (EMS2) and the feedback electromagnetic signal (EMS3) can also be considered as a sensing signal. The sensing signal can be analyzed by the sensing module 110 to obtain the at least one cardiac state (CS).

The at least one cardiac state (CS) of the present invention can be a heart rhythm or a ventricular tachycardia. Specifically, when the heart is pumping, the volume of blood in the atria or ventricles is changed with the contraction or relaxation of the heart. The volume change of blood in the atrium or ventricle will cause changes in the impedance, area, or other parameters of the planar conductor for inducing the eddy current. Therefore, different sensing signals can be used to determine the subject's heart rate, ventricular tachycardia, or other related cardiac states.

With the sensing module 110 measuring the cardiac state (CS) by using eddy current induction, it is possible to measure the cardiac state (CS) in a non-contact and a non-invasive manner. Hence, the sensing module 110 can be arranged outside the subject's body through means such as wearing. Comparing to a conventional implanted sensing mechanism, the sensing module 110 is less restricted by replacement or supplement of power required for sensing the cardiac state (CS). Therefore, the subject does not need to replace the power supply for sensing module 110. It can effectively reduce the discomfort and risk for subject to replace batteries.

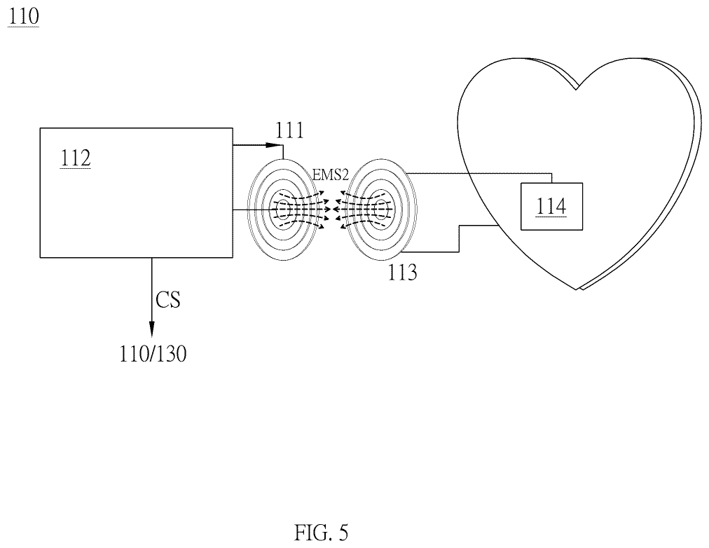

In the embodiment of the sensing module 110 with the eddy current induction, the sensitivity of the eddy current to cardiac state (CS) can be further optimized by setting a sensing receiving coil corresponding to the induction transmitting coil. Specifically, referring to FIG. 5, the sensing module 110 further includes a sensing receiving coil 113 and a sensing electrode 114. The sensing receiving coil 113 is coupled to the sensing electrode 114 and configured to receive the sensing electromagnetic signal (EMS2) to generate the feedback electromagnetic signal (EMS3). Wherein the sensing electrode 114 is arranged at a cardiac sensing area of the subject, and is configured to adjust at least one electrical parameter of the sensing receiving coil 113 for generating the feedback electromagnetic signal (EMS3) according to the at least one cardiac state (CS).

The sensing receiving coil 113 is considered as a component for generating mutual inductance to replace the ironic liquid as blood. When the sensing receiving coil 113 received the sensing electromagnetic signal (EMS2) from the sensing emitting coil 111, the sensing receiving coil 113 generates the corresponding feedback electromagnetic signal (EMS3). The sensing receiving coil 113 ensures the consistency of a location of the signal source, a frequency of the signal, and other parameters of the feedback electromagnetic signal (EMS3), thereby improving the sensing sensitivity or facilitating or simplifying the subsequent signal processing.

The sensing electrode 114 is arranged to be attached on the cardiac sensing areas that reflect heartbeats, such as the heart or near the heart. When the heart beats, the sensing electrode 114 will sense the heartbeat and accordingly change the electrical parameters such as impedance, capacitance, or inductance of the sensing electrode 114. Due to the sensing electrode 114 is electrically coupled to the sensing receiving coil 113, when the electrical parameters of the sensing electrode 114 changed due to the cardiac state (CS) (such as heart beats), the equivalent circuits of the sensing electrode 114 and the sensing receiving coil 113 will also change accordingly. The change will modulate the feedback electromagnetic signal (EMS3) generated in response to the sensing electromagnetic signal (EMS2). The modulated level (such as frequency or amplitude) of the feedback electromagnetic signal (EMS3) can be used to determine the cardiac state (CS) of the subject.

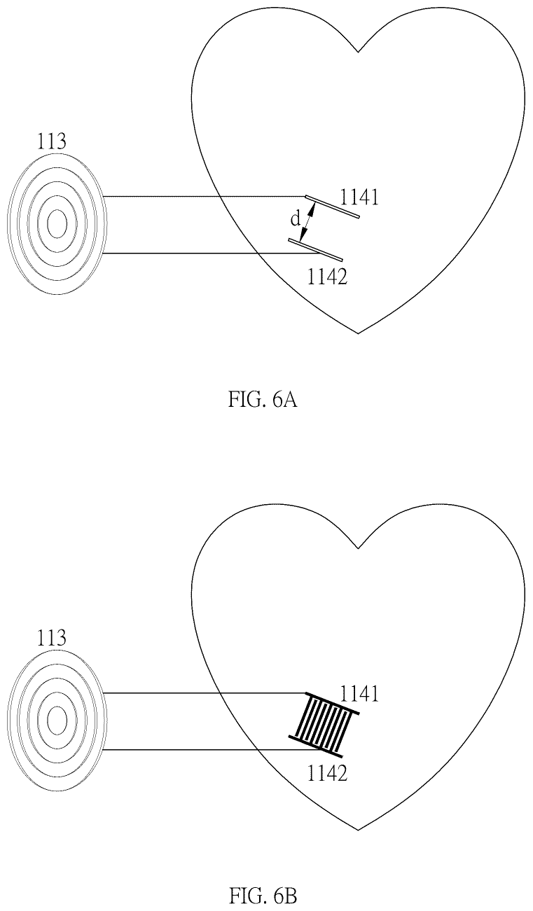

In an embodiment, referring to FIG. 6A, the sensing electrode 114 includes a first electrode 1141 and a second electrode 1142. The gap (d) between the first electrode 1141 and the second electrode 1142 is varied with the at least one cardiac state (CS). The gap (d) between the first electrode 1141 and the second electrode 1142 will change due to heartbeats, which will alter the capacitance value of the sensing electrode 114 or the dielectric value between first electrode 1141 and the second electrode 1142. The first electrodes 1141 and the second electrodes 1142 effectively reflect the cardiac state (CS). In a further example of the embodiment, referring to FIG. 6B, the first electrode 1141 and the second electrode 1142 are preferably formed as an interdigital electrode. The interdigital electrode may be referred to a comb-liked structure or a finger-liked structure arranged alternately with each other formed by the first electrode 1141 and second electrode 1142. By setting the interdigital electrode, the capacitance characteristics of the sensing electrode 114 can be further enhanced, and it can be more sensitive to the cardiac state (CS) such as heartbeats. It should be noted that the present invention is not limited to the formation of interdigital electrode, and any prior art form of interdigital electrode can be applied to the sensing electrode 114 of the present invention.

It should be noted that the sensing mechanism of the sensing electrode 114 is not limited to changes in gap distance. In an example, the sensing mechanism of the sensing electrode 114 can be measuring the electrical parameters changed by sensing the electrical signal of the cardiac state (CS). Specifically, the electrical signals of the cardiac state (CS) include nerve conduction signals, cardiac muscle discharge signals, or sinoatrial electrical signals. By using the sensing electrode 114 and the sensing receiving coil 113, the sensitivity of the eddy current measurement to the cardiac state (CS) can be improved, and the signal-to-noise ratio can be enhanced. For the sensing electrode 114 and the sensing receiving coil 113 installed inside the subject, an active power source is not required. The sensing electrode 114 and the sensing receiving coil 113 are passively changing the electrical parameters by the cardiac state (CS) and passively induced by the sensing electromagnetic signal (EMS2). The changed electrical parameters will be measured by the sensing module 110 arranged outside the subject's body. There is no need to burden the subject with the risk of replacing the sensing electrode 114 and the sensing receiving coin 113 or the battery for the sensing electrode 114 and the sensing receiving coin 113.

The corresponding number of the sensing electrode 114 of the sensing module 110 and the sensing receiving coil 113 in the embodiment is not limited to one-to-one. For example, one sensing receiving coil 113 may be correspond to multiple sensing electrodes 114. This allows for simultaneous measurement of the cardiac state (CS) for multiple positions of heart. On the other hand, multiple sensing receiving coils 113 may be correspond to multiple sensing electrodes 114, so that each sensing position can be independently measured. In addition, the sensing emitting coil 111 corresponding to the sensing receiving coil 113 can also be configured as one-to-one, many to one, or many to many with the sensing receiving coil 113. Through the above configuration, the selectivity for setting the sensing position and for the sensing electromagnetic signal (EMS2) can be improved. The divergence of sensing electromagnetic signal (EMS2) emitted by the sensing emitting coil 111 can be limited by a shielding component. For example, the shielding component can be set around the sensing emitting coil 111 to block the sensing electromagnetic signal (EMS2) and reduce the risk of affecting other circuit components, medical devices, or other sensing emitting coils 111 (if there are multiple sensing emitting coils 111).

For different subjects, the skin thickness, heart depth, and placement of the receiving coil may lead to differences efficiency in a cardiac stimulation or a cardiac state (CS) measurement. Therefore, the sensing module 110 or the stimulation module 120 can be configured to provide sensing electromagnetic signal (EMS2) or stimulation electromagnetic signal (EMS1) with different signal parameters (e.g. frequency) for different subjects. For example, the sensing module 110 or the stimulation module 120 may be configured to obtain the optimal response parameter settings based on the sensing electromagnetic signal (EMS2) or the stimulation electromagnetic signal (EMS1) with different signal parameters. In the configuration, for example, the electrical parameters of the sensing emitting coil 111 or the stimulation emitting coil 121 can be adjusted by adjustable passive components (such as capacitors), so that when the sensing emitting coil 111 or the stimulation emitting coil 121 received the an electrical signal for emitting, the sensing emitting coil 111 or the stimulation emitting coil 121 with different electrical parameters will emit different signal parameters of the stimulation electromagnetic signal (EMS1) or the sensing electromagnetic signal (EMS2). The adjustable passive components can be selected by a switch 1223 in an array manner, but are not limited to. On the other hand, the signal parameters of the electrical signal provided to the stimulation emitting coil 121 or the sensing emitting coil 111 can also be directly modulated to provide different signal parameters of the sensing electromagnetic signal (EMS2) or the stimulation electromagnetic signal (EMS1). By providing optimal response parameter settings, the measurements or the stimulation can be effectively taken to a target depth or a target location. It reduces the risk of sensing or stimulation failure or subject harm caused by excessive or insufficient energy.

In an embodiment, the sensing receiving coil 113 or the stimulation receiving coil 1231 of the present invention can be sealed with a sealing material because the sensing receiving coil 113 or the stimulation receiving coil 1231 does not require battery replacement and can be activated by the magneto-electric effect. The material selection limitation is less for the sealed sensing receiving coil 113 or stimulation receiving coil 1231. Therefore, the materials for the sensing receiving coil 113 or the stimulation receiving coil 1231 are widely selected. The sealing material may select from high biocompatibility (such as PDMS) and arranged outside layer of the sealed sensing receiving coil 113 or stimulation receiving coil 1231 for sealing. The electrical safety and biocompatibility of the sensing receiving coil 113 or the stimulation receiving coil 1231 can be improved through the sealing materials. Furthermore, the sealing materials avoids adverse reactions such as rejection in subject's body after implantation of the sensing receiving coil 113 or the stimulation receiving coil 1231.

In summary, the cardiac stimulation device 100 of the present invention includes the stimulation module 120. The stimulation module 120 stimulates a specific area(s) of the subject's heart by providing the stimulation electromagnetic signal (EMS1) from outside the patient's body. The stimulation electromagnetic signal (EMS1) causes the stimulation receiving coil 1231 to generate the stimulation voltage (SV). The stimulation electrode 1232 arranged at the subject's heart provides the stimulation voltage (SV) to the subject's heart to achieve the function of the cardiac regulation, the cardiac stimulation device 100 of the present invention further includes the sensing module 110. The sensing module 110 can be configured to measure the cardiac state (CS) based on eddy current induction. Accordingly, the cardiac stimulation device 100 of the present invention provides a non-contact measurement and a non-contact stimulation can be achieved. In addition, the stimulation receiving coil 1231 or the sensing receiving coil 113 installed in the patient's body does not require battery. Therefore, it is possible to achieve implantable cardiac stimulation and state measurement without installing any batteries in the patient's body.

The foregoing disclosure is merely preferred embodiments of the present invention and is not intended to limit the claims of the present invention. Any equivalent technical variation of the description and drawings of the present invention of the present shall be within the scope of the claims of the present invention.

What is claimed is:

1. A cardiac stimulation device, comprising:
a sensing module configured to measure at least one cardiac state of a subject; and
a stimulation module coupled to the sensing module, including:
a stimulation emitting coil;
a stimulation control circuit coupled to the stimulation emitting coil;
wherein the stimulation control circuit is configured to drive the stimulation emitting coil to emit a stimulation electromagnetic signal based on the at least one cardiac state; and
a stimulation receiver including a stimulation receiving coil and a stimulation electrode;
wherein the stimulation receiving coil is configured to receive the stimulation electromagnetic signal to generate a stimulation voltage provided to the stimulation electrode;
wherein the stimulation electrode is arranged at a cardiac stimulation area of the subject; and
wherein the stimulation voltage is provided to the cardiac stimulation area through the stimulation electrode.

2. The cardiac stimulation device of claim 1, wherein the sensing module includes:
a sensing emitting coil; and
a sensing control circuit coupled to the sensing emitting coil;
wherein the sensing control circuit is configured to drive the sensing emitting coil to emit a sensing electromagnetic signal to the subject's cardiac position, and receive a first inductive signal induced by a feedback electromagnetic signal; and
wherein the sensing control circuit is further configured to determine the at least one cardiac state based on the first inductive signal.

3. The cardiac stimulation device of claim 2, wherein the sensing module further includes:
a sensing receiving coil; and
a sensing electrode;
wherein the sensing receiving coil is coupled to the sensing electrode, and configured to receive the sensing electromagnetic signal and generate the feedback electromagnetic signal;
wherein the sensing electrode is arranged at a cardiac sensing area of the subject; and
wherein the sensing electrode is configured to adjust at least one electrical parameter of the sensing receiving coil for generating the feedback electromagnetic signal based on the at least one cardiac state.

4. The cardiac stimulation device of claim 3, wherein the sensing electrode includes a first electrode and a second electrode; and wherein a gap is formed between the first electrode and the second electrode, and the gap is varied by the at least one cardiac state.

5. The cardiac stimulation device of claim 4, wherein the first electrode and the second electrode form an interdigital electrode.

6. The cardiac stimulation device of claim 2, wherein the sensing control circuit includes a frequency adjustment unit coupled to the sensing emitting coil; and wherein frequency adjustment unit is configured to adjust a frequency of the sensing electromagnetic signal.

7. The cardiac stimulation device of claim 6, wherein the frequency adjustment unit includes a capacitor array configured to adjust an impedance of the sensing emitting coil.

8. The cardiac stimulation device of claim 2, wherein the sensing module further includes:
a shielding component at least arranged at a first side of the sensing emitting coil; wherein the shielding component at least shields a portion of the sensing electromagnetic signal emitted toward to a first direction.

9. The cardiac stimulation device of claim 1, wherein the stimulation control circuit includes:
a discharge controller configured to provide a control signal based on the at least one cardiac state;
a storage capacitor configured to storage a driving voltage; and
a switch configured to receive the control signal and enable transmitting the driving voltage to the stimulation emitting coil based on the control signal.

10. The cardiac stimulation device of claim 9, wherein the stimulation control circuit further includes:
a boost circuit configured to receive a supply voltage and boost the supplied power to the driving voltage.

11. The cardiac stimulation device of claim 1, further including:
a control module configured to receive the at least one cardiac state and provide a control signal to the stimulation module;
wherein the stimulation module is configured to emit the stimulation electromagnetic signal based on the control signal.
